SEQUENCE LISTING (15 sequences)

SEQ ID NO: 1 - DNA, 32 bp, Artificial Sequence
  Description of Artificial Sequence Synthetic primer
gaattcgccaccatggtattgggaaccataga

SEQ ID NO: 2 - DNA, 28 bp, Artificial Sequence
  Description of Artificial Sequence Synthetic primer
gcggccgcacagcacatttgaaatgccg

SEQ ID NO: 3 - DNA, 26 bp, Artificial Sequence
  Description of Artificial Sequence Synthetic primer
ggatccaactgggtgaatgtaataag

SEQ ID NO: 4 - DNA, 32 bp, Artificial Sequence
  Description of Artificial Sequence Synthetic primer
gaattcgccaccatgtgtcctcagaagctaac

SEQ ID NO: 5 - DNA, 26 bp, Artificial Sequence
  Description of Artificial Sequence Synthetic primer
agatcttacaacataaacgtctttct

SEQ ID NO: 6 - DNA, 35 bp, Artificial Sequence
  Description of Artificial Sequence Synthetic primer
gaattcgccaccatggacagcaaaggttcgtcgca

SEQ ID NO: 7 - DNA, 26 bp, Artificial Sequence
  Description of Artificial Sequence Synthetic primer
ctcgagggtggagaccacaccctggc

SEQ ID NO: 8 - DNA, 26 bp, Artificial Sequence
  Description of Artificial Sequence Synthetic primer
ggatccgcacctaacctcttgggtgg

SEQ ID NO: 9 - DNA, 28 bp, Artificial Sequence
  Description of Artificial Sequence Synthetic primer
gcggccgcttacccggagtccgggagaa

SEQ ID NO: 10 - protein, 4 aa, Artificial Sequence
  Description of Artificial Sequence Synthetic linker peptide
GGGG

SEQ ID NO: 11 - protein, 5 aa, Artificial Sequence
  Description of Artificial Sequence Synthetic linker peptide
GGGGG

SEQ ID NO: 12 - protein, 8 aa, Artificial Sequence
  Description of Artificial Sequence Synthetic linker peptide
GGGKGGGG

SEQ ID NO: 13 - protein, 8 aa, Artificial Sequence
  Description of Artificial Sequence Synthetic linker peptide
GGGNGSGG

SEQ ID NO: 14 - protein, 8 aa, Artificial Sequence
  Description of Artificial Sequence Synthetic linker peptide
GGGCGGGG

SEQ ID NO: 15 - protein, 5 aa, Artificial Sequence
  Description of Artificial Sequence Synthetic linker peptide
GPNGG

(19) United States
(12) Patent Application Publication
(10) Pub. No.: US 2006/0257361 A1
(43) Pub. Date: Nov. 16, 2006

(54) Novel form of interleukin-15, Fc-IL-15, and methods of use

(75) Inventors: Morihiro Watanabe, Bethesda, MD (US); Hiroshi Yazawa, Yamagata (JP); John Ortaldo, Frederick, MD (US); Robert Wiltrout, Woodsboro, MD (US)

Correspondence Address: EDWARDS ANGELL PALMER & DODGE LLP;(CLIENT REFERENCE NO. 47992), PO BOX 55874, BOSTON, MA, 02205, US

(73) Assignee: Government of the US, as represented by the Secretary, Department of Health and Human Services, Rockville, MD (US)

(21) Appl. No.: 11/403,681

(22) Filed: Apr. 12, 2006

Related U.S. Application Data
(60) Provisional application No. 60/670,862, filed on Apr. 12, 2005.

(51) Int. Cl. A61K 38/20; C07K 14/55; C07K 16/46

(52) U.S. Cl. 424/85.2; 424/145.1; 424/277.1; 530/351; 530/391.1

(57) ABSTRACT

The present invention relates to Fc-IL-15 hybrids, which may or may not include peptide linkers between the IL-15 and the Fc portion, for methods of treatment of tumors and viral infections. The IL-15 hybrids can be Fc-IL-15 or IL-15-Fc hybrids. The Fc-IL-15 hybrids include variants, including the IL-15 and Fc variants. The hybrids preferably (but not necessarily) include peptide linkers between the IL-15 and the Fc portion. These linkers are preferably composed of a T cell inert sequence, or any non-immunogenic sequence.

The present application claims the benefit of U.S. provisional application No. 60/670,862, filed Apr. 12, 2005, which is incorporated herein by reference in its entirety.

### FIELD OF THE INVENTION

This invention relates to novel interleukin-15 hybrid proteins, in which an interleukin-15 is conjugated with an immunoglobulin Fc, for treating various cancers and viral infections.

### BACKGROUND OF THE INVENTION

Interleukin-15 (IL-15) was initially identified as a T cell stimulatory factor {Grabstein, 1994 13/id} {Bamford, 1994 24/id} and possesses structural and functional similarity with interleukin-2 (IL-2). Although each has own α receptor, IL-15 and IL-2 shares β and γ receptor chains for signal transduction. On the other hand, recent investigation revealed that these two cytokines can be distinguished by their tissue distribution and their role in development, activation and survival of T and NK cell. While IL-2 is produced by T cells, IL-15 mRNA is expressed by a broad range of tissue including placenta, skeletal muscle, kidney, lung, heart and also multiple cell types such as activated monocytes, dendritic cells, fibroblasts {Grabstein, 1994 13/id} {Bamford, 1994 24/id} but not T cells {Tagaya, 1996 25/id}. IL-15 inhibits IL-2-mediated activation-induced cell death of lymphocytes {Marks-Konczalik, 2000 12/id} and stimulates memory CD8⁺ T cell proliferation {Lau, 1994 26/id} {Zhang, 1998 30/id} {Murali-Krishna, 1999 27/id} {Swain, 1999 28/id}. NK cells are not detected in the mice that are deficient of IL-15 gene {Kennedy, 2000 22/id} or either of IL-15α {Lodolce, 1998 23/id}, β {Suzuki, 1997 32/id} or γ {DiSanto, 1995 33/id} receptor genes while IL-2−/− mice do possess inducible NK cell activity{Kundig, 1993 31/id}. It was also shown that IL-15 differentiates NK precursor to mature NK cell in the presence of bone marrow stroma{Yu, 1998 21/id} suggesting a critical role of IL-15 in NK cell development.

Due to the immune stimulating properties of IL-15, it is not surprising that this protein promotes anti-tumor activities in NK-dependent {Suzuki, 2001 17/id} {Kobayashi, 2004 36/id}or T cell dependent{Hazama, 1999 34/id} {Meazza, 2000 18/id} {Klebanoff, 2004 37/id} manner. IL-15 is also important for protecting virus infection, expansion and maintenance of T cell response in immunization and development of dendritic cell. All these immunomodulatory activities of IL-15 suggest that this cytokine can be an attractive therapeutic reagent for various diseases where host immune system play either promoting or detrimental role.

On the other hand, in vivo studies to characterize the effect of IL-15 have been hampered because of limited availability of recombinant protein and also low efficiency of IL-15 secretion by native gene. Indeed, IL-15 was previously studied mainly by IL-15 transgenic, IL-15−/− or IL-15Rα −/− mice or by using IL-15 expressing tumor cells. Hence biological effect of IL-15 in therapeutic settings is largely unknown.

Most cytokines, including IL-15, have relatively short circulation half-lives since they are produced in vivo to act locally and transiently. To use IL-15 as an effective systemic therapeutic, one needs relatively large doses and frequent administrations. Such frequent parenteral administrations are inconvenient and painful. Further, toxic side effects are associated with IL-15 administration are so severe that some cancer patients cannot tolerate the treatment. These side effects are probably associated with administration of a high dosage.

To overcome these disadvantages, one can modify the molecule to increase its circulation half-life or change the drug's formulation to extend its release time. The dosage and administration frequency can then be reduced while increasing the efficacy. Immunoglobulins of IgG and IgM class are among the most abundant proteins in the human blood. They circulate with half-lives ranging from several days to 21 days. IgG has been found to increase the half-lives of several ligand binding proteins (receptors) when used to form recombinant hybrids, including the soluble CD4 molecule, LHR, and the IFN-γ receptor (Mordenti J. et al., Nature, 337:525-31, 1989; Capon, D. J. and Lasky, L. A., U.S. Pat. No. 5,116,964; Kurschner, C et al., J. Immunol. 149:4096-4100, 1992).

### SUMMARY OF THE INVENTION

The present invention relates to Fc-IL-15 hybrids, which may or may not include peptide linkers between the IL-15 and the Fc portion, for methods of treatment of tumors and viral infections. The IL-15 hybrids can be Fc-IL-15 or IL-15-Fc hybrids. The components of Fc-IL-15 hybrid include variants, including IL-15 variants and Fc. The hybrids preferably (but not necessarily) include peptide linkers between the IL-15 and the Fc portion. These linkers are preferably composed of a T cell inert sequence, or any non-immunogenic sequence. The preferred Fc fragment is a human immunoglobulin Fc fragment, preferably the γ4 chain.

In one embodiment, the C-terminal end of the IL-15 is linked to the N-terminal end of the Fc fragment. An additional IL-15 (or other cytokine) can attach to the N-terminal end of any other unbound Fc chains in the Fc fragment, resulting in a homodimer, if the Fc selected is the γ4 chain. If the Fc fragment selected is another chain, such as the μ chain, then, because the Fc fragments form pentamers with ten possible binding sites, this results in a molecule with interleukin, or another cytokine, linked at each of ten binding sites

The two moieties of the hybrid are preferably linked through a T cell immunologically inert peptide including, for example, peptides with Gly Ser repeat units. Because these peptides are immunologically inactive, their insertion at the fusion point eliminates any neoantigenicity which might have been created by the direct joining of the Fc-IL-15 moieties.

The Fc-IL-15 hybrids of the invention are predicted to have a much longer half-life in vivo than the native IL-15, and this is supported by experimental data. Cytokines are generally small proteins with relatively short half-lives which dissipate rapidly among various tissues, including at undesired sites. It is believed that small quantities of some cytokines can cross the blood-brain barrier and enter the central nervous system, thereby causing severe neurological toxicity. The FC-IL-15 hybrids of the present invention would be especially suitable for treating tumors, including melanoma and renal cell carcinoma, because these products will have a long retention time in the vasculature and will not penetrate undesired sites.

### BRIEF DESCRIPTION OF THE DRAWINGS

FIG. 1 contains graphic representations A) of IL-15 plasmids. E: enhancer of cytomegalovirus immediate early gene, EF-1α: human elongation factor-1α promoter, pA: polyA signal from bovine growth hormone, Fc: constant region of mouse IgG1, IL-15: mature form of human IL-15 cDNA, P: signal sequence from bovine preprolactin, S: signal sequence from mouse IL-12 p40; B) Production of IL-15 in transient transfection medium of 293 cell and C) its effect on the proliferation CTLL-2 for 72 hours.

FIG. 2 contains graphic representations of the pharmacokinetics of gene products in the serum; wherein A) Hydrodynamic delivery of pPR-IL-15 (▴), pIL 15-Fc (▾) or pFc (▪) plasmids: B) Hydrodynamic delivery of pIL-2 (♦) o r pFc (▪) plasmid; C) Induction of IFN-γ in the serum by the delivery of pPR-IL15 (▴), pFc-IL15 (▾), pIL-2 (♦) or pFc (▪) plasmids.

FIG. 3 contains graphic representations of the kinetics of NK cells in liver (▴), spleen (B) and lung (C) of Balb/c mice after delivery of either pPR-IL15 or pFc-IL15 hydrodynamics; Kinetics of NK cell counts in liver (D), spleen (E) and lung (F) after delivery of pIL-2 by hydrodynamic gene delivery.

FIG. 4 is a graphic representation of the augmentation of NK cell cytotoxicity after either pPR-IL15, pFc-IL15 or pIL-2 hydrodynamic gene delivery.

FIG. 5 contains graphic representations of anti-tumor activities of IL-15 gene delivery. 2×10⁵ of Renca cells were injected intravenously into Balb/c mice. 3 days after, plasmids in the figure were injected by hydrodynamic gene delivery; Dose of plasmid delivered: pPR-IL-15, pFc-IL15 and pFc: 10 g per mouse. Pil-2: 2 weeks after the tumor injection, all the mice were terminated and the number of nodules in the lung was counted.

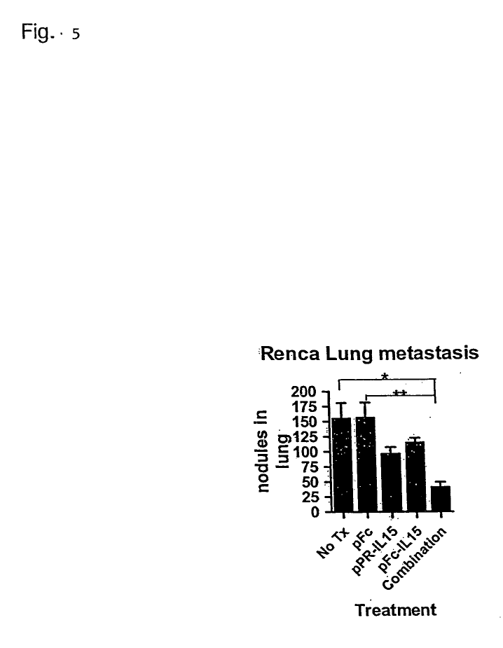

### DETAILED DESCRIPTION OF THE INVENTION

We have now demonstrated inter alia cytotoxic activity and therapeutic potential of IL-15 for preexisting tumor metastasis by augmenting killing activities of NK cell. This was made possible by using unique IL-15 plasmids and highly efficient system to deliver them to subjects including animal models.

Recent findings suggest that IL-15 is trans-presented in association with its high affinity α on NK or T cells to transmit its signal through the rest of the receptor complex, β and γ subunits that are expressed on the target cells such as DC, macrophage to. In fact expressing IL-15Rα in tumor cells effectively elicit NK cell mediated anti-tumor response ( ). This new paradigm would prompt unconventional strategies for IL-15 to elicit its biological activities effectively in comparison with the other cytokines. However, there lies a technical challenge to utilize this theory to deliver IL-15. In addition, exogenous IL-15 produces its biological activities {Kennedy, 2000 22/id}. Therefore, the current study attempted to improve secretion of IL-15 by modification of its cDNA sequence was either by replacing its signal sequence {Marks-Konczalik, 2000 12/id} or by creating fusion protein. Employing strong promoter, CMV enhancer plus EF-1α promoter {Kobayashi, 1997 20/id} and as an efficient system for in vivo gene transfer, hydrodynamic gene delivery is employed. Subsequently, concentration of IL-15 after pPR-IL15 and pFc-IL15 delivery is more than 1 ng/ml and 10 ng/ml, respectively over 6 days in the mouse serum. This concentration is the same as or 2-12 times higher than the serum concentration of IL-15 transgenic mouse (150-800 pg/ml) {Marks-Konczalik, 2000 12/id}, (186.7±41.8 pg/ml) {Fehniger, 2001 8/id}. This successful delivery of IL-15 gene resulted in the increase of NK, NKT and T in liver is consistent with transgenic mouse study. While the number of these cells reached maximal 4 days after the pIL-2 injection and all the cell type disappear quickly, IL-15 gene delivery continuously increased these cells for a week, Disappearance of IL-2 protein in three days after the pIL-2 gene delivery might explain this kinetics. The other possibility of this rapid decrease can be IL-2.

The Fc Protein:

Immunoglobulins of IgG class are among the most abundant proteins in human blood. Their circulation half-lives can reach as long as 21 days. Fusion proteins have been reported to combine the Fc regions of IgG with the domains of another protein, such as various cytokines and soluble receptors (see, for example, Capon et al., Nature, 337:525-531, 1989; Chamow et al., Trends Biotechnol., 14:52-60, 1996); U.S. Pat. Nos. 5,116,964 and 5,541,087). The prototype fusion protein is a homodimeric protein linked through cysteine residues in the hinge region of IgG Fc, resulting in a molecule similar to an IgG molecule without the CH1 domains and light chains. Due to the structural homology, Fc fusion proteins exhibit in vivo pharmacokinetic profile comparable to that of human IgG with a similar isotype. To extend the circulating half-life of IL-15 and/or to increase its biological activity, it is desirable to make fusion proteins containing IL-15 linked to the Fc portion of the human IgG protein as disclosed or described in this invention.

The term “Fc” refers to molecule or sequence comprising the sequence of a non-antigen-binding fragment resulting from digestion of whole antibody, whether in monomeric or multimeric form. The original immunoglobulin source of the native Fc is preferably of human origin and may be any of the immunoglobulins, although IgG1 and IgG2 are preferred, Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. International applications WO 97/34631 (published Sep. 25, 1997) and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by other means.

The Il-15 Protein:

Interleukin 15 is constitutively produced in animals (Tough and Sprent, J. Exp. Med. 179:1127 (1994); Zhang et al., Immunity 8:591 (1998); Peschon et al., J. Exp. Med. 180:1955 (1994); Moore et al., J. Immunol. 157:2366 (1996); Sudo et al., J. Exp. Med. 170:333 (1989); Heufler et al., J. Exp. Med. 178:1109 (1993); Doherty et al., J. Immunol. 1556:735 (1996); Jonuleit et al., J. Immunol. 158:2610 (1997); Tagaya et al., Proc. Natl. Acad. Sci. USA 94:14444 (1997); Bamford et al., J. Immunol. 160:4418 (1998)). Although IL-2 is not constitutively produced in animals, recent evidence suggests that it is present, even in young pathogen free mice, retained on the extracellular matrix (Wrenshall and J. Immunol. 163:3793 (1999)). This may be the source of the IL-2 which is functioning in the experiments reported here. Interleukin-2 can induce activated T cells to die (Zheng et al., J. Immunol. 160:763 (1998); Refaeli et al., Immunity, 8:615 (1998)) and/or, as illustrated by the experiments reported here, kill proliferating CD8+ memory phenotype cells (but see Ke et al., J. Exp. Med. 187:49 (1998)). IL-2 or IL-2R.alpha. deficient mice suffer from lymphoproliferative diseases, especially if infected (Kramer et al., Eur. J. Immunol. 24:2317 (1994); Simpson et al., Eur. J. Immunol. 25:2618 (1995); Willerford et al., Immunity 3:521 (1995); Kung et al., Cell. Immunol. 185:158 (1998); Erhardt et al., J. Immunol. 158:566 (1998)). Without being bound by theory, the present inventors suggest that this is because lack of IL-2 allows unchecked proliferation of memory T cells in response to IL-15 in these animals.

Mice deficient in IL-15R-α lack CD8+ memory phenotype T cells (Lodolce et al., Immunity 9:669 (1998)) and IL-15, induced by poly IC or interferon, makes CD8+ T cells of memory phenotype divide (Tough and Sprent, J. Exp. Med. 179:1127 (1994); Zhang et al., Immunity 8:591 (1998); Tough et al., Science 272:1947 (1996)). Competition for IL-15 may, in fact, limit the total number of CD8+ memory CD8+ T cells the animal can sustain (Selin et al., J. Exp. Med. 183:2489 (1996)). Conversely, production of IL-2 during an immune response may check otherwise uncontrolled responses by bystander CD8+ memory T cells induced by increased levels of IL-15.

The Fc-IL-15 Hybrid Protein:

Any “linker” group is optional. When present, its chemical structure is not critical, since it serves primarily as a spacer. The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly).sub.4, (Gly).sub.5), poly(Gly-Ala), and polyalanines. Other specific examples of linkers are:
(Gly).sub.3 Lys(Gly).sub.4 (SEQ ID NO: 333);
(Gly).sub.3 AsnGlySer(Gly).sub.2 (SEQ ID NO: 334);
(Gly).sub.3 Cys(Gly).sub.4 (SEQ ID NO: 335); and
GlyProAsnGlyGly (SEQ ID NO: 336).

To explain the above nomenclature, for example, (Gly).sub.3 Lys(Gly).sub.4 means Gly-Gly-Gly-Lys-Gly-Gly-Gly-Gly. Combinations of Gly and Ala are also preferred. The linkers shown here are exemplary; linkers within the scope of this invention may be much longer and may include other residues.

Uses of the Fc-IL-15 Hybrid Protein:

In immune responses the stimulatory effects of one process are frequently counterbalanced by the inhibitory effects of another. Such contrary effects allow the immune system to respond vigorously but not uncontrollably to infections. The opposing effects of IL-15 and IL-2 reported here represent another example of the checks and balances inherent in the mechanisms of immunity.

Accordingly, one embodiment of the present invention relates to a composition and method for increasing a desirable immune response, and particularly, for enhancing T cell memory in an individual. For example, it is desirable to increase (e.g., enhance, upregulate, stimulate, activate) T cell memory responses in a patient that has cancer (i.e., increase memory T cell responses against a tumor antigen), in a patient with an infectious disease (i.e., increase memory T cell responses against a pathogen, such as a virus or bacterium), and/or in a patient that has an immunodeficiency disease (i.e., increase memory T cell responses against a variety of antigens). Other diseases and conditions in which it is desirable to increase T cell memory will be apparent to those of skill in the art and are intended to be encompassed by the present invention, including the prevention of opportunistic infection.

Preferably, the memory T cell response is enhanced by administering to the patient a composition comprising at least one agent that increases the activity of IL-15 in the patient and/or at least one agent that decreases the activity of IL-2 in the patient. In a preferred embodiment, both agents are administered together in a composition with or without an antigen against which the memory T cell response is to be increased. When the composition of the present invention is administered in conjunction with an antigen (an immunogen), the composition of the present invention serves as a vaccine adjuvant, to enhance the development of a memory T cell response against the antigen. In a particularly preferred embodiment, the administration of the composition is targeted to a particular site or cell in a patient (e.g., a site of a tumor, an organ that is infected with a pathogen), so that the effect of the composition is substantially localized to the T cells for which increased response is desired.

Administration of Fc-IL-15 Hybrid Protein/Nucleotides:

A composition of IL-15 hybrid protein includes compositions containing a pharmaceutically acceptable carrier, which includes pharmaceutically acceptable excipients and/or delivery vehicles, for delivering the agent(s) to a patient. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is the site of delivery of the composition of the present invention, including a vaccination site, the site of a tumor, the site of an autoimmune reaction, and/or a specific tissue or cell (e.g., a tumor cell, a graft cell, a memory T cell, a CD25⁺ T cell). Preferred pharmaceutically acceptable carriers are capable of maintaining a protein, antibody, small molecule, or recombinant nucleic acid molecule useful in the present invention in a form that, upon arrival of the protein, antibody or recombinant nucleic acid molecule at the cell target in a culture or in patient, the protein, antibody or recombinant nucleic acid molecule is capable of interacting with its target (e.g., a cell).

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises an agent of the present invention (e.g., a protein (including homologues), an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible).

A pharmaceutically acceptable carrier which is capable of targeting can be referred to as a “delivery vehicle” or more particularly, a “targeting delivery vehicle.” Delivery vehicles of the present invention are capable of delivering a composition of the present invention to a target site in a patient. A “target site” refers to a site in a patient to which one desires to deliver a composition (e.g., a memory T cell, a CD25⁺ T cell, a tumor site/cell, a site of an immune response, a vaccination site, a tissue/cell graft). For example, a target site can be any cell which is targeted by direct injection or delivery using liposomes, viral vectors or other delivery vehicles, including ribozymes. A cell or tissue can be targeted, for example, by including in the vehicle a targeting moiety, such as a ligand capable of selectively (i.e., specifically) binding another molecule at a particular site (i.e., a molecule on the surface of the target cell or a molecule expressed by cells in the target tissue/organ). Examples of such ligands include antibodies, antigens, receptors and receptor ligands. Alternatively, particular modes of administration (e.g., direct injection) and/or types of delivery vehicles (e.g., liposomes) can be used to deliver a composition preferentially to a particular site (see, for example, the use of cationic liposomes by intravenous delivery to target pulmonary tissues, described below).

Examples of delivery vehicles include, but are not limited to, artificial and natural lipid-containing delivery vehicles, viral vectors, and ribozymes. Natural lipid-containing delivery vehicles include cells and cellular membranes. Artificial lipid-containing delivery vehicles include liposomes and micelles. A delivery vehicle of the present invention can be modified to target to a particular site in a mammal, thereby targeting and making use of a compound of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other suitable delivery vehicles include gold particles, poly-L-lysine/DNA-molecular conjugates, and artificial chromosomes.

In one embodiment, an agent of the present invention is targeted to a target site by using an antibody that selectively binds to a protein expressed on the surface of the target cell. For example, an antibody could bind to a tumor cell antigen or to an autoantigen. Such an antibody can include functional antibody equivalents such as antibody fragments (antigen binding fragments) (e.g., Fab fragments or Fab.sub.2 fragments) and genetically-engineered antibodies, including single chain antibodies or chimeric antibodies, including bi-specific antibodies that can bind to more than one epitope. Such targeting antibodies are complexed with an agent that increases or decreases the activity of IL-15 action of the cell or in the local environment of the cell that is targeted, and serves to deliver the agent to the preferred site of action. The antibodies can be complexed to the target by any suitable means, including by complexing with a liposome, or by recombinant or chemical linkage of the agent to the antibody. In one embodiment, the agent is a second antibody or portion thereof that forms a chimeric or bispecific antibody with the targeting antibody.

When the agent is a nucleic acid molecule, a host cell is preferably transfected in vivo (i.e., in a mammal) as a result of administration to an animal of a recombinant nucleic acid molecule, or ex vivo, by removing cells from the animal and transfecting the cells with a recombinant nucleic acid molecule ex vivo. Transfection of a nucleic acid molecule into a host cell according to the present invention can be accomplished by any method by which a nucleic acid molecule administered into the cell in vivo or ex vivo, and includes, but is not limited to, transfection, electroporation, microinjection, lipofection, adsorption, viral infection, naked DNA injection and protoplast fusion. Methods of administration are discussed in detail below.

It may be appreciated by one skilled in the art that use of recombinant DNA technologies can improve expression of transfected nucleic acid molecules by manipulating, for example, the duration of expression of the gene (i.e., recombinant nucleic acid molecule), the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, increasing the duration of expression of the recombinant molecule, substitutions or modifications of transcription control signals (e.g., promoters, operators, enhancers), substitutions or modifications of translational control signals (e.g., ribosome binding sites, Shine-Dalgamo sequences), modification of nucleic acid molecules of the present invention to correspond to the codon usage of the host cell, and deletion of sequences that destabilize transcripts. The activity of an expressed recombinant protein of the present invention may be improved by fragmenting, modifying, or derivatizing nucleic acid molecules encoding such a protein.

In one embodiment of the present invention, a recombinant nucleic acid molecule useful in the present invention is administered to a patient in a liposome delivery vehicle, whereby the nucleic acid sequence encoding the protein enters the host cell (i.e., the target cell) by lipofection. A liposome delivery vehicle contains the recombinant nucleic acid molecule and delivers the molecules to a suitable site in a host recipient. According to the present invention, a liposome delivery vehicle comprises a lipid composition that is capable of delivering a recombinant nucleic acid molecule of the present invention, including both plasmids and viral vectors, to a suitable cell and/or tissue in a patient. A liposome delivery vehicle of the present invention comprises a lipid composition that is capable of fusing with the plasma membrane of the target cell to deliver the recombinant nucleic acid molecule into a cell.

A liposome delivery vehicle of the present invention can be modified to target a particular site in a mammal (i.e., a targeting liposome), thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands). Suitable liposomes for use with the present invention include any liposome. Preferred liposomes of the present invention include those liposomes commonly used in, for example, gene delivery methods known to those of skill in the art. Complexing a liposome with a nucleic acid molecule of the present invention can be achieved using methods standard in the art.

In accordance with the present invention, acceptable protocols to administer an agent including the route of administration and the effective amount of an agent to be administered to an animal can be determined and executed by those skilled in the art. Effective dose parameters can be determined by experimentation using in vitro cell cultures, in vivo animal models, and eventually, clinical trials if the patient is human. Effective dose parameters can be determined using methods standard in the art for a particular disease or condition that the patient has or is at risk of developing. Such methods include, for example, determination of survival rates, side effects (i.e., toxicity) and progression or regression of disease.

Administration routes include in vivo, in vitro and ex vivo routes. In vivo routes include, but are not limited to, oral, nasal, intratracheal injection, inhaled, transdermal, rectal, and parenteral routes. Preferred parenteral routes can include, but are not limited to, subcutaneous, intradermal, intravenous, intramuscular and intraperitoneal routes. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. Intravenous, intraperitoneal, intradermal, subcutaneous and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention with a carrier capable of withstanding degradation by digestive enzymes in the gut of an animal. Examples of such carriers, include plastic capsules or tablets, such as those known in the art. Direct injection techniques are particularly useful for suppressing graft rejection by, for example, injecting the composition into the transplanted tissue, or for site-specific administration of an agent, such as at the site of a tumor. Administration of a composition locally within the area of a target cell/tissue (e.g., transplanted tissue or tumor) refers to injecting or otherwise introducing the composition centimeters and preferably, millimeters within the target cell/tissue. Such routes can include the use of pharmaceutically acceptable carriers as described above.

Ex vivo refers to performing part of the regulatory step outside of the patient, such as by transfecting a population of cells removed from a patient with a recombinant molecule comprising a nucleic acid sequence encoding IL-2 or IL-15 according to the present invention under conditions such that the recombinant molecule is subsequently expressed by the transfected cell, or contacting a cell with another agent useful in the invention, and returning the transfected/contacted cells to the patient. In vitro and ex vivo routes of administration of a composition to a culture of host cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety. For example, using liposome delivery, U.S. Pat. No. 5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. As discussed above, Liu et al., 1997, ibid. demonstrated that intravenous delivery of cholesterol-containing cationic liposomes containing genes preferentially targets pulmonary tissues and effectively mediates transfer and expression of the genes in vivo. Several publications by Dzau and collaborators demonstrate the successful in vivo delivery and expression of a gene into cells of the heart, including cardiac myocytes and fibroblasts and vascular smooth muscle cells using both naked DNA and Hemagglutinating virus of Japan-liposome delivery, administered by both incubation within the pericardium and infusion into a coronary artery (intracoronary delivery) (See, for example, Aoki et al., 1997, J. Mol. Cell, Cardiol. 29:949-959; Kaneda et al., 1997, Ann N.Y. Acad. Sci. 811:299-308; and von der Leyen et al., 1995, Proc Natl Acad Sci USA 92:1137-1141). Delivery of numerous nucleic acid sequences has been accomplished by administration of viral vectors encoding the nucleic acid sequences. Using such vectors, successful delivery and expression has been achieved using ex vivo delivery (See, of many examples, retroviral vector; Blaese et al., 1995, Science 270:475-480; Bordignon et al., 1995, Science 270:470-475), nasal administration (CFTR-adenovirus-associated vector), intracoronary administration (adenoviral vector and Hemagglutinating virus of Japan, see above), intravenous administration (adeno-associated viral vector; Koeberl et al., 1997, Proc Natl Acad Sci USA 94:1426-1431). A publication by Maurice et al., 1999, ibid. demonstrated that an adenoviral vector encoding a .beta.2-adrenergic receptor, administered by intracoronary delivery, resulted in diffuse multichamber myocardial expression of the gene in vivo, and subsequent significant increases in hemodynamic function and other improved physiological parameters. Levine et al. describe in vitro, ex vivo and in vivo delivery and expression of a gene to human adipocytes and rabbit adipocytes using an adenoviral vector and direct injection of the constructs into adipose tissue (Levine et al., 1998, J. Nutr. Sci. Vitaminol. 44:569-572). Gene delivery to synovial lining cells and articular joints has had similar successes. Oligino and colleagues report the use of a herpes simplex viral vector which is deficient for the immediate early genes, ICP4, 22 and 27, to deliver and express two different receptors in synovial lining cells in vivo (Oligino et al., 1999, Gene Ther. 6:1713-1720). The herpes vectors were administered by intraarticular injection. Kuboki et al. used adenoviral vector-mediated gene transfer and intraarticular injection to successfully and specifically express a gene in the temporomandibular joints of guinea pigs in vivo (Kuboki et al., 1999, Arch. Oral. Biol. 44:701-709). Apparailly and colleagues systemically administered adenoviral vectors encoding IL-10 to mice and demonstrated successful expression of the gene product and profound therapeutic effects in the treatment of experimentally induced arthritis (Apparailly et al., 1998, J. Immunol. 160:5213-5220). In another study, murine leukemia virus-based retroviral vector was used to deliver (by intraarticular injection) and express a human growth hormone gene both ex vivo and in vivo (Ghivizzani et al., 1997, Gene Ther. 4:977-982). This study showed that expression by in vivo gene transfer was at least equivalent to that of the ex vivo gene transfer. As discussed above, Sawchuk et al. has reported successful in vivo adenoviral vector delivery of a gene by intraarticular injection, and prolonged expression of the gene in the synovium by pretreatment of the joint with anti-T cell receptor monoclonal antibody (Sawchuk et al., 1996, ibid. Finally, it is noted that ex vivo gene transfer of human interleukin-1 receptor antagonist using a retrovirus has produced high level intraarticular expression and therapeutic efficacy in treatment of arthritis, and is now entering FDA approved human gene therapy trials (Evans and Robbins, 1996, Curr. Opin. Rheumatol. 8:230-234). Therefore, the state of the art in gene therapy has led the FDA to consider human gene therapy an appropriate strategy for the treatment of at least arthritis. Taken together, all of the above studies in gene therapy indicate that delivery and expression of a cytokine-encoding recombinant nucleic acid molecule according to the present invention is feasible.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include a nucleic acid molecule of the present invention, and preferably includes a recombinant molecule of the present invention that preferably is replication, or otherwise amplification, competent.

According to the method of the present invention, an effective amount of an agent that regulates IL-15 to administer to an animal comprises an amount that is capable of regulating IL-15 activity, and preferably effecting a modulation of an immune response at a target site, without being toxic to the animal. An amount that is toxic to an animal comprises any amount that causes damage to the structure or function of an animal (i.e., poisonous). A preferred single dose of an agent typically comprises between about 0.01 microgram X kilogram⁻¹ and about 10 milligram X kilogram⁻¹ body weight of an animal. A more preferred single dose of an agent comprises between about 1 microgram X kilogram⁻¹ and about 10 milligram X kilogram⁻¹ body weight of an animal. An even more preferred single dose of an agent comprises between about 5 microgram times kilogram⁻¹ and about 7 milligram X kilogram⁻¹ body weight of an animal. An even more preferred single dose of an agent comprises between about 10 microgram X kilogram⁻¹ and about 5 milligram X kilogram⁻¹ body weight of an animal. A particularly preferred single dose of an agent comprises between about 0.1 milligram X kilogram⁻¹ and about 5 milligram X kilogram⁻¹ body weight of an animal, if the an agent is delivered by aerosol. Another particularly preferred single dose of an agent comprises between about 0.1 microgram X kilogram.sup⁻¹ and about 10 microgram X kilogram⁻¹ body weight of an animal, if the agent is delivered parenterally. These doses particularly apply to the administration of protein agents, antibodies, and/or small molecules (i.e., the products of drug design). Preferably, a protein or antibody of the present invention is administered in an amount that is between about 50 U/kg and about 15,000 U/kg body weight of the patient. When the compound to be delivered is a nucleic acid molecule, an appropriate single dose results in at least about 1 pg of protein expressed per mg of total tissue protein per μg of nucleic acid delivered. More preferably, an appropriate single dose is a dose which results in at least about 10 pg of protein expressed per mg of total tissue protein per .mu.g of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered. A preferred single dose of a naked nucleic acid vaccine ranges from about 1 nanogram (ng) to about 100 μg, depending on the route of administration and/or method of delivery, as can be determined by those skilled in the art.

The methods of the present invention can be used in any animal, and particularly, in any animal of the Vertebrate class, Mammalia, including, without limitation, primates, rodents, livestock and domestic pets. Preferred mammals to treat using the method of the present invention include humans.

The following examples are given for the purpose of illustrating various embodiments of the invention and are not meant to limit the present invention in any fashion.

All documents mentioned herein are incorporated herein by reference in their entirety.

### EXAMPLES

The following materials and methods were used in the Examples:

Mice

Balb/c female mice at 6-8 weeks of age were obtained from the National Cancer Institute at Frederick and maintained under specific pathogen-free conditions. Animal care was provided in accordance with the procedures outlined in the Guide for the Care and Use of Laboratory Animals (NIH Pub. No. 86-23, 1985).

Cell Culture

The human embryonic kidney cell line, 293, was maintained in Dulbecco Modified Eagle Medium with 10% FCS. The Renca mouse renal cell carcinoma cell line and YAC-1 mouse lymphoma cell line was passed in Balb/c mice or was cultured in 10% FBS RPMI 1640, 10 mM sodium pyruvate, 2 mM L-glutamine, 50 U/ml penicillin and 50 mg/ml streptomycin. Medium for CTLL-2 was Dulbecco Modified Eagle Medium with 10% FCS and 20 IU/ml of human interleukin-2.

Construction of Expression Vectors for IL-15 and IL-2

Gene fragments to construct expression plasmid was generated by PCR by using following primer pairs: full length IL-15 sense:

GAATTCGCCACCATGGTATTGGGAACCATAGA, anti-sense:
GCGGCCGCACAGCACATTTGAAATGCCG, mature forms of IL-15, sense:
GGATCCAACTGGGTGAATGTAATAAG, signal sequence of mouse interleukin-12 p40, sense: GAATTCGCCACCATGTGTCCTCAGAAGCTAAC, antisense:
AGATCTTACAACATAAACGTCTTTCT and bovine preprolactin sense:
GAATTCGCCACCATG GACAGCAAAG GTTCGTCGCA, anti-sense:
CTCGAGGGTGGAGACCACACCCTGGC, mouse IgG2a Fc
GGATCCGCACCTAACCTCTTGGGTGG, antisense:
GCGGCCGCTTACCCGGAGTCCGGGAGAA. Underlined sequences are restriction enzyme sites added to each primer sequence. Italicized GCCACC is Kozak sequence, to facilitate effective gene expression. For construction of IL-2 expression vector, mouse full length IL-2 cDNA was used. As a vector cDNA for constant region of mouse immunoglobulin preceded by IL-12 p40 signal sequence was used. All these five vectors are under a control of CMV enhancer and human elongation factor-1α promoter.
   Transfection

1×10⁶ 293 cells were plated per well in a six-well plate. Twenty-four hours later, 2 μg of plasmid DNA was transfected with lipofectamin 2000 (Invitrogen, Carlsbad, Calif.) according to the manufacturer's protocol.

DNA Array

Equal amounts of total RNA were analyzed by DNA array (GEArray Q Series Mouse Angiogenesis Gene Array, Super Array, Bethesda, Md.) according to the manufacturer's instructions.

ELISA

Human IL-15, mouse IL-2 or mouse IFN-γ in either mouse serum or transfection supernatant were measured by Quantikine ELISA Kit (R&D system, Minneapolis Minn.) according to instructions provided by manufacture.

In Vivo Gene Transfer by Hydrodynamic Gene Delivery

Plasmid DNA was prepared using an Endofree Mega Kit (Qiagen, Valencia, Calif.). The endotoxin concentration of these plasmid preparations was less than 0.1 endotoxin unit/μg of DNA. Plasmids were injected into mice by hydrodynamic gene delivery as described {Zhang, 1999 6/id}. In brief, five micrograms of plasmid DNA in 1.6 ml 0.9% NaCl was injected through tail vein of mice in approximately 5 seconds using a 27 gauge needle.

Cell Preparation From Liver, Spleen and Lung.

After mice were euthenized, their portal veins were flushed by HBSS and the livers were removed from mice, minced with Stomacker. After several wash, leukocytes from liver were isolated using 40-80% Percoll (Amersham, Piccadilly. N.J.) gradient. Spleens were squeezed and the resultant splenocytes were washed twice with HBSS. Harvested lung was cut with scissors to a size of less than 1 mm diameter. The minced lung tissues were digested with 0.03% of Collagenase I and 0.14% of DNase I (Sigmna, St Louis, Mo.) at 37° C. for 1 hours. Debris was removed by strainer and leukocytes were recovered as an interface of Picoll 40-80% gradient.

Cytotoxicity Assay

Cytolytic activity was tested as described {Watanabe, 1999 5/id)}. In belief, leukocytes and ⁵¹Cr-labeled Yac-1 cells were co-cultured in U-bottom 96-well plate for 4 hours in triplicate. Lytic unit was calculated based on the ⁵¹Cr release in the culture supernatant.

Flowcytometry

Antibodies used for flowcytometry were the following: DX5, NK1.1, CD3, CD4, CD8, CD94, Ly49.

Treatment of Mice with Lung or Liver Metastasis

Balb/c mice were injected with 1×10⁵ of Renca intravenously through tail vein for lung metastasis model or 5×10⁴ of Renca intrasplenically for liver metastasis. 5 minutes after intrasplenic Renca injection, spleen was removed. 3 days after the tumor challenge, mice were injected with plasmid by hydrodynamic gene delivery. All mice were terminated 2 weeks after Renca cell injection to count the number of metastasis in lung and liver.

### EXAMPLE 1

Modified Forms of IL-15 Expression Vector Efficiently Produce Biologically Active IL-15 In Vitro

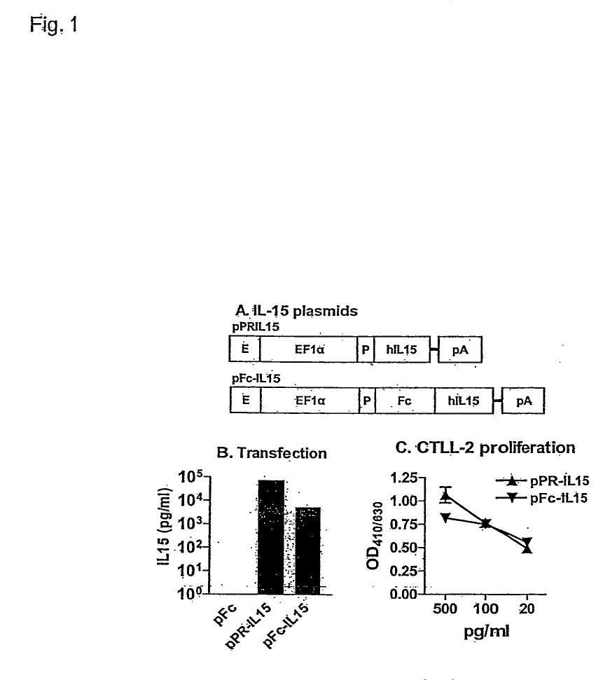

Although mRNA of IL-15 is detected at high level in multiple tissues and cell types, IL-15 protein is poorly translated and secreted. Three primary posttranscriptional checkpoints are responsible for this observation: Translation of IL-15 is impeded by multiple AUGs in the 5′ untranslated region, inefficient long signal peptides (LSPs) and short signal peptides {Onu, 1997 15/id} {Tagaya, 1997 16/id}, and a negative regulator near the COOH terminus. In fact, when IL-15 plasmid, which encodes IL-15 full cDNA sequence, is transfected into 293 cells, 10-30 pg/ml of IL-15 was detected in the supernatant. Therefore, to explore the efficient expression system for mammalian cells, we constructed following 4 different IL-15 expression vectors by replacing its own signal sequence or adding constant fragment of mouse immunoglobulin G2a.: (1) mature form of IL-15 with bovine preprolactin signal sequence (bPRL) {Marks-Konczalik, 2000 12/id}, designates as pPRIL15, (2) mature form of IL-15 proceeded by mouse immunoglobulin constant region (mFc) with bPRL-pFc-IL15, (3) mature form of IL-15 followed by mFc with PRL pIL15-Fc, (4) mature form of IL-15 with mouse interleukin-12 p40 signal sequence pP40-IL15 (FIG. 1A). Since human IL-15 cross-reacts with mouse and only human IL-15 ELISA was commercially available, human IL-15 cDNA was used to construct these vectors. When these vectors were transiently transfected into 293 cells, pPR-L15 secreted 73.5 ng/ml of IL-15 in the supernatant as highest among the IL-15 vectors constructed above (FIG. 1B). The rest of the plasmid produced 3-5 ng/ml of IL-15 in the supernatant (FIG. 1B). To verify these IL-15 proteins encoded by the plasmids are biologically active, these supernatants were tested for proliferation of mouse T cell line CTLL-2. These supernatants, which contain 20-500 ng/ml of IL-15, stimulated the growth of CTLL-2 in dose dependent manner in 72 hours (FIG. 1C). Thus, modified forms pf expression plasmids for IL-15 were constructed and these plasmids produce biologically active IL-15 more efficiently than the vector encoding original IL-15 full cDNA sequence.

### EXAMPLE 2

Hydrodynamic Gene Delivery of pPR-IL15 and pFc-IL15 Produced High Concentration of Systemic IL-15 and Induced Interferon-γ In Vivo

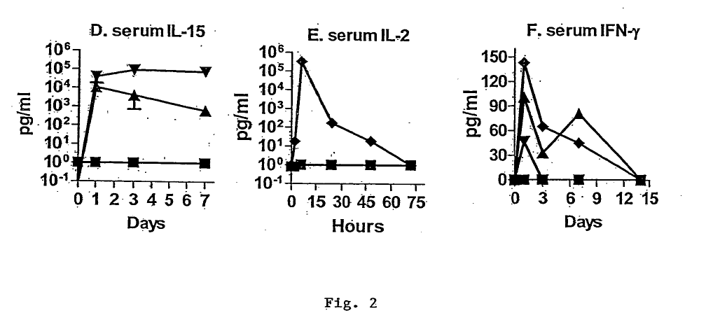

Hydrodynamic gene delivery is highly efficient method to transfer naked DNA into mouse {Zhang, 1999 6/id}. In this gene delivery system, expression of transgene is almost exclusively in mouse liver {Liu, 1999 7/id} and several cytokine genes were successfully delivered to mice {He, 2000 2/id} {Jiang, 2001 3/id} {Chen, 2003 1/id}. Therefore, these novel IL-15 constructs were transferred in vivo by hydrodynamic delivery to investigate systemic production of IL-15. The pPR-IL-15 and pFc-IL15 was injected into mice to investigate pharmacokinetics of their gene products. 24 hours after pPR-IL15 injection, the production of IL-15 in the serum was 10.9 ng/ml and then gradually decreased over 7 days to 0.6 ng/ml (FIG. 2A). Serum concentration of Fc-IL15 also quickly increased to 39.5 ng/ml within 24 hours pFc-IL15, elevated slightly to 90.5 ng/ml at 72 hours and maintained this concentration for another 4 days (FIG. 2A). The peak of IL-15 production by pIL15-Fc was almost 100 times less than either pPRIL-15 or pFc-IL-15.35-1400 pg/ml of IL-15 was detected in supernatant of tumor cells that were stably transduced with IL-15 gene {Suzuki, 2001 17/id} {Meazza, 2000 18/id} {Di Carlo, 2000 19/id}. Particularly hydrodynamic delivery of pPR15 and pFc-IL15 resulted in 10.6 ng/ml and 39.5 ngml of IL-15 in the mouse serum and these concentrations are 3-10 times as high as observed in transgenic mice where expression of IL-15 was driven by MHC class I promoter {Fehniger, 2001 8/id}. Therefore, the production of IL-15 in vivo is quite efficient particularly when pPR-IL15 and pFc-IL15 was delivered by hydrodynamic injection. IL-15 Because of its biological similarity with IL-15, pIL-2, an expression plasmid for mouse IL-2 was also delivered by hydrodynamics. Serum concentration of IL-2 was 333 ng/ml at 8 hours after the pIL-2 injection by hydrodynamic gene delivery and the serum level of IL-2 gradually decreased to undetectable by ELISA with 72 hours (FIG. 2B).

As a marker to test biological activity of these gene products, induction of IFN-γ was also measured. Serum concentration of IFN-γ was maximal at 24 hours in all treatments (FIG. 2C). This peak was followed by quick drop in the next 48 hours (FIG. 2C). Thus, it was demonstrated that biologically active IL-15 protein was produced in vivo at high efficiency by hydrodynamic injection of IL-15 plasmids. Because of efficient production of IL-15, the following studies used only pPRIL-15 and pFc-IL 15.

### EXAMPLE 3

Hydrodynamic Delivery of IL-15 Plasmids Increased NK, NKT and T Cell in In Vivo

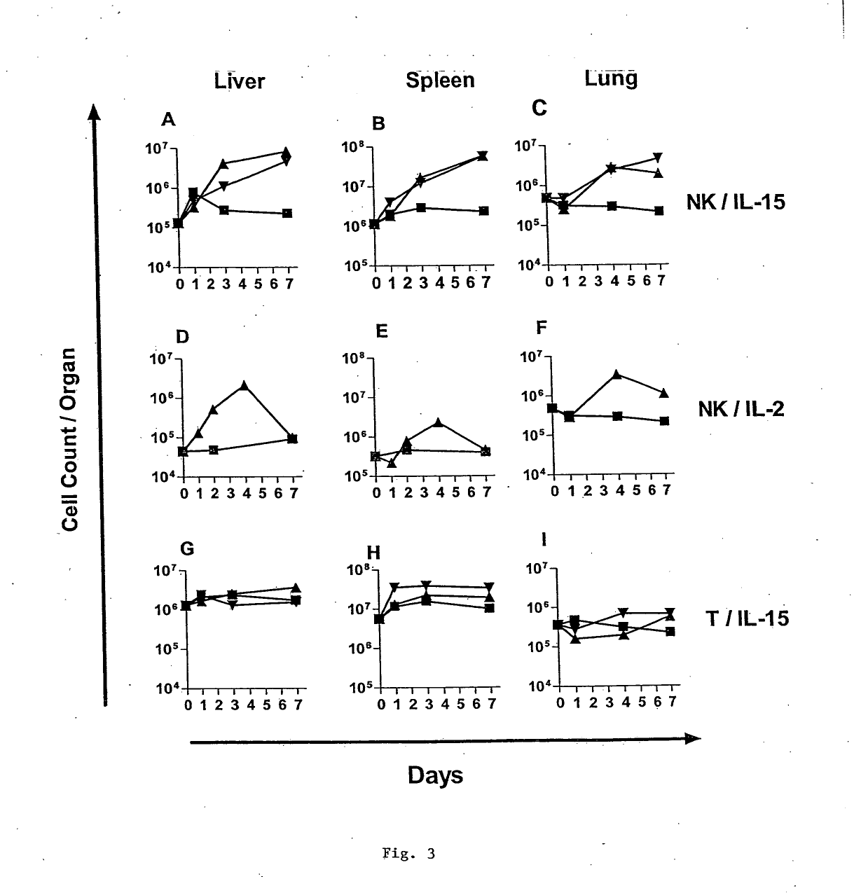

In IL-15 transgenic mice, there is an increase of NK, NKT and T cells in immune organs {Fehniger, 2001 8/id} {Marks-Konczalik, 2000 12/id}. Since high concentration of IL-15 protein was achieved in mouse serum by hydrodynamic delivery, dynamics of these immune cells were investigated in liver, spleen and lung. The total number of liver leukocytes increased to 7- and 3.8 fold in 7 days by single injection of pPRIL-15 and pFc-IL-15, respectively (FIG. 3A). The impact of these gene deliveries on DX5+CD3−NK cells in the liver were more pronounced and there were 62 fold increase by pPR-IL15 and 35 fold increase by pFc-IL15 in 7 days. pPR-IL15 was also effective on the expansion of NKT (DX5⁺ CD3⁺) cells and T lymphocytes (FIG. 3A). The same trend was observed in spleen (FIG. 3B) except pFc-IL15 hydrodynamic delivery was almost as effective as pPR-IL15 in total leukocyte, NK and NKT cell number and was more effective in expansion of T lymphocytes than pPR-IL15 (FIG. 3B). There was slight increase of each cell population by the pFc delivery (FIGS. 3A and B). In mice treated with pIL-2 hydrodynamic gene delivery, expansion of liver and spleen NK cell became most prominent 4 days after the injection and returned approximately to pretreatment level on day 7 and spleen (FIG. 3C). Delivery of IL-15 plasmids to C57 resulted in the similar increase as observed in Balb/c mice in the liver and spleen. On the other hand, the peak of NK, NKT and T cells was on day 4, not day 7 of this assay. To further characterize the subpopulation of NK, expression of Ly49 was investigated. The kinetics of ly49 positive cells was paralell to the total NK (FIG. 3D) in liver, spleen and lung. In summary, in vivo transfer of IL-15 plasmids resulted in dramatic and continuous increase of NK, NKT and T cells in liver, spleen over seven days. On the other hand, the peak of these cells in lung is day 4, followed by gradual decrease. IL-2 gene therapy, in contrast, caused distinctive kinetics of subpopulation of these cells from IL-15 and displays rapid and dramatic increase up to 4 days and quickly dropped to almost pretreatment level by 7 days after the injection.

### EXAMPLE 4

Hydrodynamic Delivery of pPR-IL15 Augmented Cytotoxicity Particularly in Lung

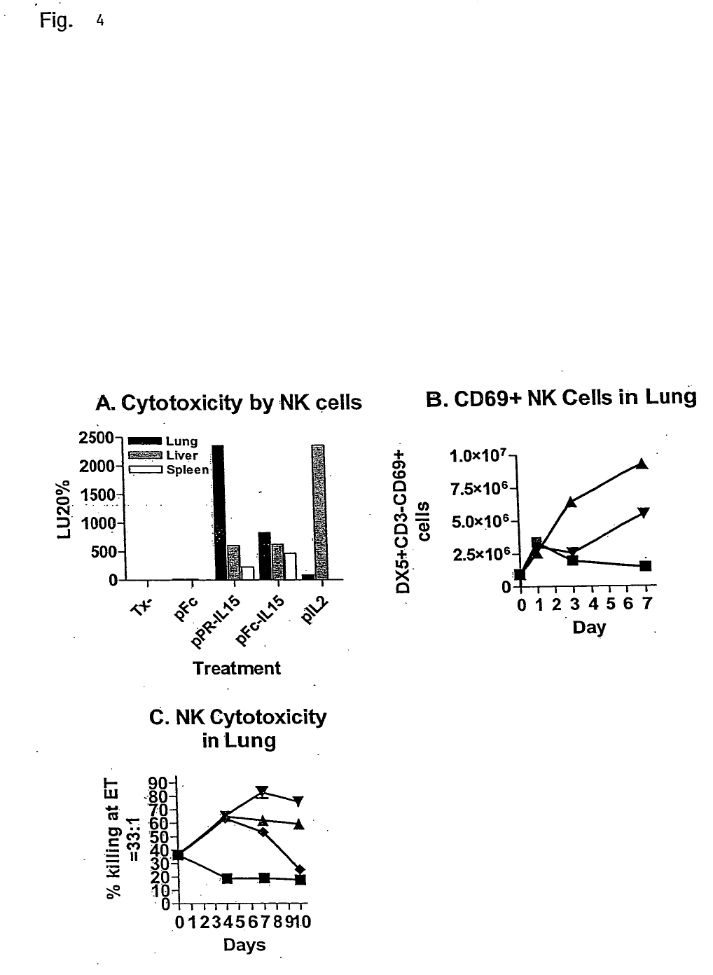

To test if the NK cells expanded by IL-15 hydrodynamic gene delivery is functional, leukocytes recovered from the liver, spleen and lung 4 days after either single injection of pPR-IL15, recombinant human IL-15 protein IL-2. The leukocytes particularly from the lung displayed 42-fold increase of lytic unit, which represents killing activity of given number of cells, by pPR-IL15 hydrodynamic gene delivery (FIG. 4A). This result was consistent with the recombinant IL-15 or IL-2 protein treatment. In the same experiment, the number of NK cells increased 2-5 fold by these treatments (FIG. 4B). In contrast, in the liver, there were pronounced increase of NK cells.

### EXAMPLE 5

Inhibition of Renca Metastasis in Lung by pPR-IL15, pFc-IL15 or Their Combination

IL-15 stimulates NK cells to elicit anti-tumor activities in prevention tumor models. Since IL-15 gene therapy resulted in dramatic increase of cytotoxic activity particularly in the lung, we tested the anti-metastatic activities of pPR-IL15, pFc-IL15. As a tumor model, murine renal cell carcinoma Renca was injected intravenously in order to develop lung metastasis and treatment was initiated 3 days later. In 14 days, approximately 200 of visible metastasis was formed in the lung without any treatment. This pulmonary metastasis was inhibited 30%, 50% and 70% by delivery of pPR-IL15, pFc-IL15 or their combination, respectively. pIL-2 treatment did not suppressed the metastatic growth of tumor. Although the same treatments were applied to 3 day liver metastasis model, no inhibitory effect was observed in IL-15 gene therapies. Delivery of pIL-2, however, inhibited metastasis formation in 2 weeks by 50%. To summarize, IL-15 and IL-2 gene therapy has differential effect to suppress established, early stage metastasis model in lung and liver.

### EXAMPLE 6

Comparison of DNA Profile in Liver NK Cells from IL-15 and IL-2 Treated Mice.

To examine the different mechanisms between IL-15 and IL-2, we observed apoptosis signal cascade in NK cells treated with each cytokines. BIRC4, Caspase 3, lymphotoxin beta receptor TNFSF14 and TNFSF9 levels were elevated in IL-2 treatment group but TRAF1 was up-regulated in IL-15.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

The following specific references, also incorporated herein by reference in their entirety, are indicated in the Examples and discussion above by a number in parentheses or other indication.
1. Grabstein, K. H., Eisenman, J., Shanebeck, K., Rauch, C., Srinivasan, S., Fung, V., Beers, C., Richardson, J., Schoenborn, M. A., Ahdieh, M., and. Cloning of a T cell growth factor that interacts with the beta chain of the interleukin-2 receptor. Science, 264: 965-968, 1994.
2. Bamford, R. N., Grant, A. J., Burton, J. D., Peters, C., Kurys, G., Goldman, C. K., Brennan, J., Roessler, E., and Waldmann, T. A. The interleukin (IL) 2 receptor beta chain is shared by IL-2 and a cytokine, provisionally designated IL-T, that stimulates T-cell proliferation and the induction of lymphokine-activated killer cells. Proc. Natl. Acad. Sci. U.S.A, 91: 4940-4944, 1994.
3. Tagaya, Y., Bamford, R. N., DeFilippis, A. P., and Waldmann, T. A. IL-15: a pleiotropic cytokine with diverse receptor/signaling pathways whose expression is controlled at multiple levels. Immunity., 4: 329-336, 1996.
4. Marks-Konczalik, J., Dubois, S., Losi, J. M., Sabzevari, H., Yamada, N., Feigenbaum, L., Waldmann, T. A., and Tagaya, Y. IL-2-induced activation-induced cell death is inhibited in IL-15 transgenic mice. Proc. Natl. Acad. Sci. U.S.A, 97: 11445-11450, 2000.
5. Lau, L. L., Jamieson, B. D., Somasundaram, T., and Ahmed, R. Cytotoxic T-cell memory without antigen. Nature, 369: 648-652, 1994.
6. Zhang, X., Sun, S., Hwang, I., Tough, D. F., and Sprent, J. Potent and selective stimulation of memory-phenotype CD8+ T cells in vivo by IL-15. Immunity, 8: 591-599, 1998.
7. Murali-Krishna, K., Lau, L. L., Sambhara, S., Lemonnier, F., Altman, J., and Ahmed, R. Persistence of memory CD8 T cells in MHC class I-deficient mice. Science, 286: 1377-1381, 1999.
8. Swain, S. L., Hu, H., and Huston, G. Class II-independent generation of CD4 memory T cells from effectors. Science, 286: 1381-1383, 1999.
9. Kennedy, M. K., Glaccum, M., Brown, S. N., Butz, E. A., Viney, J. L., Embers, M., Matsuki, N., Charrier, K., Sedger, L., Willis, C. R., Brasel, K., Morrissey, P. J., Stocking, K., Schuh, J. C. L., Joyce, S., and Peschon, J. J. Reversible Defects in Natural Killer and Memory CD8 T Cell Lineages in Interleukin 15-deficient Mice. J. Exp. Med., 191: 771-780, 2000.
10. Lodolce, J. P., Boone, D. L., Chai, S., Swain, R. E., Dassopoulos, T., Trettin, S., and Ma, A. IL-15 receptor maintains lymphoid homeostasis by supporting lymphocyte homing and proliferation. Immunity., 9: 669-676, 1998.
11. Suzuki, H., Duncan, G. S., Takimoto, H., and Mak, T. W. Abnormal Development of Intestinal Intraepithelial Lymphocytes and Peripheral Natural Killer Cells in Mice Lacking the IL-2 Receptor beta □Chain. J. Exp. Med., 185: 499-506, 1997.
12. DiSanto, J. P., Muller, W., Guy-Grand, D., Fischer, A., and Rajewsky, K. Lymphoid Development in Mice with a Targeted Deletion of the Interleukin 2 Receptor {gamma} Chain. PNAS, 92: 377-381, 1995.
13. Kundig, T. M., Schorle, H., Bachmann, M. F., Hengartner, H., Zinkemagel, R. M., and Horak, I. Immune responses in interleukin-2-deficient mice. Science, 262: 1059-1061, 1993.
14. Yu, H., Fehniger, T. A., Fuchshuber, P., Thiel, K. S., Vivier, E., Carson, W. E., and Caligiuri, M. A. Flt3 Ligand Promotes the Generation of a Distinct CD34+ Human Natural Killer Cell Progenitor That Responds to Interleukin-15. Blood, 92: 3647-3657, 1998.
15. Suzuki, K., Nakazato, H., Matsui, H., Hasumi, M., Shibata, Y., Ito, K., Fukabori, Y., Kurokawa, K., and Yamanaka, H. NK cell-mediated anti-tumor immune response to human prostate cancer cell, PC-3: immunogene therapy using a highly secretable form of interleukin-15 gene transfer. J. Leukoc. Biol., 69: 531-537, 2001.
16. Kobayashi, H., Dubois, S., Sato, N., Sabzevari, H., Sakai, Y., Waldmann, T. A., and Tagaya, Y. The role of trans-cellular IL-15-presentation in the activation of NK-mediated killing, which leads to enhanced tumor immunesurveillance. Blood, 2003-2012, 2004.
17. Hazama, S., Noma, T., Wang, F., Iizuka, N., Ogura, Y., Yoshimura, K., Inoguchi, E., Hakozaki, M., Hirose, K., Suzuki, T., and Oka, M. Tumour cells engineered to secrete interleukin-15 augment anti-tumour immune responses in vivo. Br. J. Cancer, 80: 1420-1426, 1999.
18. Meazza, R., Lollini, P. L., Nanni, P., De Giovanni, C., Gaggero, A., Comes, A., Cilli, M., Di Carlo, E., Ferrini, S., and Musiani, P. Gene transfer of a secretable form of IL-15 in murine adenocarcinoma cells: effects on tumorigenicity, metastatic potential and immune response. Int. J. Cancer, 87: 574-581, 2000.
19. Klebanoff, C. A., Finkelstein, S. E., Surman, D. R., Lichtman, M. K., Gattinoni, L., Theoret, M. R., Grewal, N., Spiess, P. J., Antony, P. A., Palmer, D. C., Tagaya, Y., Rosenberg, S. A., Waldmann, T. A., and Restifo, N. P. IL-15 enhances the in vivo antitumor activity of tumor-reactive CD8+T cells. Proc. Natl. Acad. Sci. U.S.A, 101: 1969-1974, 2004.
20. Ashkar, A. A. and Rosenthal, K. L. Interleukin-15 and natural killer and NKT cells play a critical role in innate protection against genital herpes simplex virus type 2 infection. J. Virol., 77: 10168-10171, 2003.
21. Mohamadzadeh, M., Berard, F., Essert, G., Chalouni, C., Pulendran, B., Davoust, J., Bridges, G., Palucka, A. K., and Banchereau, J. Interleukin 15 skews monocyte differentiation into dendritic cells with features of Langerhans cells. J. Exp. Med., 194: 1013-1020, 2001.
22. Zhang, G., Budker, V., and Wolff, J. A. High levels of foreign gene expression in hepatocytes after tail vein injections of naked plasmid DNA. Hum. Gene Ther., 10: 1735-1737, 1999.
23. Watanabe, M., Fenton, R. G., Wigginton, J. M., McCormick, K. L., Volker, K. M., Fogler, W. E., Roessler, P. G., and Wiltrout, R. H. Intradermal delivery of IL-12 naked DNA induces systemic NK cell activation and Th1 response in vivo that is independent of endogenous IL-12 production. J. Immunol., 163: 1943-1950, 1999.
24. Onu, A., Pohl, T., Krause, H., and Bulfone-Paus, S. Regulation of IL-15 secretion via the leader peptide of two IL-15 isoforms. J. Immunol., 158: 255-262, 1997.
25. Tagaya, Y., Kurys, G., Thies, T. A., Losi, J. M., Azimi, N., Hanover, J. A., Bamford, R. N., and Waldmann, T. A. Generation of secretable and nonsecretable interleukin 15 isoforms through alternate usage of signal peptides. PNAS, 94: 14444-14449, 1997.
26. Liu, F., Song, Y., and Liu, D. Hydrodynamics-based transfection in animals by systemic administration of plasmid DNA. Gene Ther., 6: 1258-1266, 1999.
27. He, Y., Pimenov, A. A., Nayak, J. V., Plowey, J., Falo, L. D., Jr., and Huang, L. Intravenous injection of naked DNA encoding secreted flt3 ligand dramatically increases the number of dendritic cells and natural killer cells in vivo. Hum. Gene Ther., 11: 547-554, 2000.
28. Jiang, J., Yamato, E., and Miyazaki, J. i. Intravenous Delivery of Naked Plasmid DNA for in Vivo Cytokine Expression. Biochemical and Biophysical Research Communications, 289: 1088-1092, 2001.
29. Chen, H. W., Lee, Y. P., Chung, Y. F., Shih, Y. C., Tsai, J. P., Tao, M. H., and Ting, C. C. Inducing long-term survival with lasting anti-tumor immunity in treating B cell lymphoma by a combined dendritic cell-based and hydrodynamic plasmid-encoding IL-12 gene therapy. Int. Immunol., 15: 427-435, 2003.
30. Di Carlo, E., Comes, A., Basso, S., De Ambrosis, A., Meazza, R., Musiani, P., Moelling, K., Albini, A., and Ferrini, S. The Combined Action of IL-15 and IL-12 Gene Transfer Can Induce Tumor Cell Rejection Without T and NK Cell Involvement. J Immunol, 165: 3111-3118, 2000.
31. Fehniger, T. A., Suzuki, K., Ponnappan, A., VanDeusen, J. B., Cooper, M. A., Florea, S. M., Freud, A. G., Robinson, M. L., Durbin, J., and Caligiuri, M. A. Fatal leukemia in interleukin 15 transgenic mice follows early expansions in natural killer and memory phenotype CD8+ T cells. J. Exp. Med., 193: 219-231, 2001.
32. Yajima, T., Nishimura, H., Wajjwalku, W., Harada, M., Kuwano, H., and Yoshikai, Y. Overexpression of interleukin-15 in vivo enhances antitumor activity against MHC class I-negative and -positive malignant melanoma through augmented NK activity and cytotoxic T-cell response. Int. J. Cancer, 99: 573-578, 2002.
33. Dubois, S., Mariner, J., Waldmann, T. A., and Tagaya, Y. IL-15Ralpha recycles and presents IL-15 In trans to neighboring cells. Immunity., 17: 537-547, 2002.
34. Burkett, P. R., Koka, R., Chien, M., Chai, S., Chan, F., Ma, A., and Boone, D. L. IL-15R alpha expression on CD8+ T cells is dispensable for T cell memory. Proc. Natl. Acad. Sci. U.S.A, 100: 4724-4729, 2003.
35. Burkett, P. R., Koka, R., Chien, M., Chai, S., Boone, D. L., and Ma, A. Coordinate expression and trans presentation of interleukin (IL)-15Ralpha and IL-15 supports natural killer cell and memory CD8+ T cell homeostasis. J Exp. Med., 200: 825-834, 2004.
36. Koka, R., Burkett, P. R., Chien, M., Chai, S., Chan, F., Lodolce, J. P., Boone, D. L., and Ma, A. Interleukin (IL)-15R[alpha]-deficient natural killer cells survive in normal but not IL-15R[alpha]-deficient mice. J Exp. Med., 197: 977-984, 2003.
37. Koka, R., Burkett, P., Chien, M., Chai, S., Boone, D. L., and Ma, A. Cutting edge: murine dendritic cells require IL-15R alpha to prime NK cells. J Immunol., 173: 3594-3598, 2004.
38. Naora, H. and Gougeon, M. L. Enhanced survival and potent expansion of the natural killer cell population of HIV-infected individuals by exogenous interleukin-15. Immunol. Lett., 68: 359-367, 1999.
39. Kobayashi, M., Tanaka, A., Hayashi, Y., and S S The CMV enhancer stimulates expression of foreign genes from the human EF-1alpha promoter. Anal. Biochem., 247: 179-181, 1997.

We claim:

1. A method of treatment for a patient having cancer comprising the administration to a patient in need of such treatment a pharmaceutically effective amount of Fc-IL-15.

2. A method of claim 1 wherein the cancer is melanoma.

3. A method of claim 1 wherein the cancer is renal cell carcinoma.

4. A method of claim 1 wherein a patient is selected for treatment on the basis of a diagnosis of cancer and the Fc-IL-15 is administered to the selected patient.

5. A method of claim 4 wherein the cancer is melanoma.

6. A method of claim 4 wherein the cancer is renal cell carcinoma.

7. A method of treatment for a patent having a condition involving uncontrolled cell proliferation comprising the administration to a patient in need of such treatment a pharmaceutically effective amount of Fc-IL-15.

8. A vaccine comprising a vaccinating antigen and Fc-IL-15.

9. The vaccine of claim 8 wherein the vaccinating antigen is selected from the group consisting of: a tumor antigen and an antigen from an infectious disease pathogen.

10. A method of increasing T lymphocyte memory comprising administering to a patient a vaccine of claim 8.

11. A method of enhancing vaccination efficiency comprising the co-administration of Fc-Il-15 as a vaccine adjuvant.

12. A fusion protein comprising a natural Interleukin-15 polypeptide linked to the Fc portion of an immunoglobulin.

13. A fusion protein of claim 1 wherein the immunoglobulin is IgG.

14. An isolated nucleic acid that encodes a fusion protein of claims 1 or 2.

15. A modified Interleukin-15 having a longer in vivo half-life than a natural, unmodified Interleukin-15.

16. A fusion protein of claim 12 wherein the Fc portion is linked to the N-terminus of natural IL-15.

17. A fusion protein of claim 12 wherein the Fc portion is linked to the C-terminus of natural IL-15.

18. A method of preventing an opportunistic infection in a patient comprising the administration to a patient in need of such prevention a pharmaceutically effective amount of Fc-IL-15.
